(19) United States
(12) Patent Application Publication
(10) Pub. No.: US 2015/0292045 A1
(43) Pub. Date: Oct. 15, 2015

(54) ALTERNATIVE USES FOR HBV ASSEMBLY EFFECTORS

(71) Applicant: INDIANA UNIVERSITY RESEARCH AND TECHNOLOGY CORPORATION, Indianapolis, IN (US)

(72) Inventors: Massimo Levrero, Rome (IT); Adam Zlotnick, Bloomington, IN (US)

(21) Appl. No.: 14/441,621

(22) PCT Filed: Nov. 8, 2013
(86) PCT No.: PCT/US13/69280

§ 371 Date: May 8, 2015

Related U.S. Application Data
(60) Provisional application No. 61/724,800, filed on Nov. 9, 2012.

(51) Int. Cl. C12Q 1/70; A61K 31/4453; A61K 31/506

(52) U.S. Cl. CPC C12Q 1/706; A61K 31/506; A61K 31/4453; C12Q 2600/158; C12Q 2600/136

(57) ABSTRACT

Described herein are methods for identifying compounds useful for the treatment of infection by hepatitis B virus (HBV).

### CROSS-REFERENCE TO RELATED APPLICATIONS

This application claims the benefit of U.S. Provisional Application No. 61/724,800, filed Nov. 9, 2012, the disclosure of which is incorporated by reference herein in its entirety.

### TECHNICAL FIELD

The present invention relates to methods for identifying compounds useful for the treatment of infection by hepatitis B virus (HBV).

### BACKGROUND AND SUMMARY OF THE INVENTION

The hepatitis B virus (HBV), which belongs to the hepadnavirus family, is a causative agent of acute and chronic hepatitis. HBV infections are the world's ninth leading cause of death. HBV infection often leads to acute hepatitis and liver damage, and causes abdominal pain, jaundice, and elevated blood levels of certain enzymes. HBV can cause fulminant hepatitis, a rapidly progressive form of the disease in which massive sections of the liver are destroyed. Many patients recover from acute viral hepatitis, but in certain other patients, especially young children, viral infection persists for an extended, or indefinite, period, causing a chronic infection. Chronic infections can lead to chronic persistent hepatitis. Chronic persistent hepatitis can cause fatigue, liver damage, cirrhosis of the liver, and hepatocellular carcinoma, a primary liver cancer.

HBV infection is a serious problem among the homo- and heterosexual population, intravenous drug users, organ transplant recipients, and blood transfusion patients. New infection with HBV can be prevented by vaccination. However, the present vaccination is not effective for the approximately 350 million chronic carriers worldwide. It has been observed that suppression or eradication of the replication of HBV in the liver leads to improved liver pathology and decreased progression to liver cirrhosis and hepatocellular carcinoma.

One of the current therapies approved in the United States for treating chronic hepatitis B infection is alpha interferon, which is far from ideal. According to the American Liver Foundation and the International Hepatitis Foundation, patients with conditions such as advanced hepatitis, HIV co-infection, drug abuse or others are not eligible for this treatment, resulting in less than 50% of chronic carriers obtaining this therapy. Of these patients, only about 40% respond to the treatment. Many of these patients also relapse after treatment is stopped, and only about 30% of the patients show a long term benefit. Viral disappearance is only seen in about 10-20% of the treated patients. These data suggest that there is an extremely low response rate in patients treated with alpha interferon. In addition to the low response rate, interferon therapy causes severe side effects such as insomnia, depression, nausea, vomiting, fever and fatigue. Another approved class of drugs for treating HBV infection is reverse transcriptase inhibitors exemplified by lamivudine, entecavir, and tenofovir. Although reverse transcriptase inhibitors have good antiviral activity, resistance can develop rapidly during treatment, there is cross-reactivity of resistance, and side effects such as kidney damage. There is also cross-reactivity between reverse transcriptase inhibitors for HBV and HIV. Furthermore, reverse transcriptase inhibitors are not known to lead to HBV clearance and, worse, discontinuation of the therapy is known to lead to a rebound effect occurs in most cases that can be life threatening.

The development of novel combination based therapies for HBV infection requires new antivirals that block viral life cycle functions other than those associated with the viral polymerase. The HBV Core, that comprises the viral capsid, nucleic acid, and host and viral ancillary proteins, represents an attractive target. Proper assembly of the capsid is critical for RNA packaging, reverse transcription, and intracellular trafficking. It is believed that normal assembly is nucleated by a trimer of Cp dimers and proceeds without accumulating observable populations of intermediates. Moreover, core proteins (Cp) have been shown to interact with histones and to bind the nuclear circular covalently closed DNA (cccDNA), possibly contributing to the regulation of cccDNA function and the maintenance of the cccDNA stability. Hetero-aryl-dihydropyrimidines (HAPs) are a class of antivirals which inhibit HBV replication in vitro and in vivo (Deres, Science 2003; Zlotnick, PNAS 2007). HAPs enhance the rate and the extent of core protein (Cp) assembly over a broad concentration range and act as allosteric effectors to induce an assembly-active state or, at high concentration, stabilize preferentially non-capsid polymers of Cp interfering with normal virion assembly, resulting in an antiviral effect. Core proteins (Cp) have been shown to interact with histones and to bind the nuclear cccDNA, possibly contributing to the regulation of cccDNA function and the maintainance of the cccDNA stability (Bock, JMB 2001; Pollicino, Gastroenterology 2006; Guo, Epigenetics 2011). Described herein is the discovery that at higher concentrations, above where DNA synthesis is blocked, HAPs interfere with production of viral RNA.

In one illustrative embodiment of the invention, a method is described for treating a patient having an infection by hepatitis B virus (HBV), the method comprising the step of administering to the patient a therapeutically effective amount a compound capable of inhibiting accumulation of HBV pregenomic RNA (pgRNA) in an HBV infected cell of the patient.

In another embodiment, a method is described for identifying a compound useful for the treatment of infection hepatitis B virus (HBV), comprising: contacting a cell infected with HBV with a test compound in a culture medium, or administering a potential compound to an animal; retrieving a sample from the cell, the culture medium, or from tissue of the animal, at one or more time points; analyzing the sample for one or more attributes selected from the group consisting of HBV cccDNA concentration, amount of methylated cccDNA, acetylation state of cccDNA, HBV cccDNA transcription, HBV RNA concentration in cellular cytoplasm, HBV RNA concentration in the cell nucleus, concentration of unassembled capsid protein, and HBV S antigen concentration; and identifying the compound as useful for treating hepatitis B based on the reduction or increase of one of more of the attributes.

### BRIEF DESCRIPTION OF THE DRAWINGS

FIG. 1 HAPs: a new class of antivirals inhibiting HBV replication HAPs misdirect HBV capsid assembly and block HBV replication.

FIG. 2 HAP12 targets cccDNA transcription (1)HAP12 treatment induces a complete suppression of HBV replication at 72 and 96 hrs with a peak >50% reduction of pgRNA transcription at 96 hours and an approximate 30% decrease in cccDNA levels.

a) In HepG2 cells transfected with monomeric linear full length HBV pgRNA and HBV replication are dependent on cccDNA formation, chromatinization and transcription. b) Cytoplasmic HBV core particles were isolated from untreated and HAP12-treated cells at the indicated time points after transfection. Results are expressed as number of HBV DNA copies per transfected cells. c) cccDNA accumulation in untreated and HAP12-treated HepG2 cells transfected with wild type HBV genomes. qPCR analysis was performed using selective cccDNA primers to amplify cccDNA and beta-globin primers to normalize the DNA samples. Results are expressed as number of cccDNA copies per transfected cells. d) mRNAs were prepared from untreated and IFNα-treated HepG2 cells transfected with wild type HBV genomes and harvested at the indicated times post-transfection. Specific primers were utilized to quantify the HBV pregenomic RNA and GAPDH amplification was used to normalize for equal loading of each RNA sample. All histograms show mean values from two independent experiments; bars indicate standard deviations (SD)

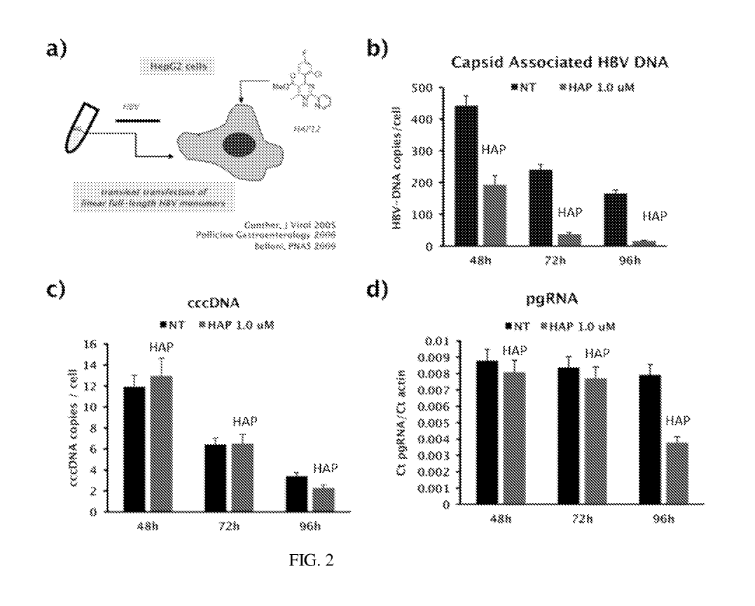

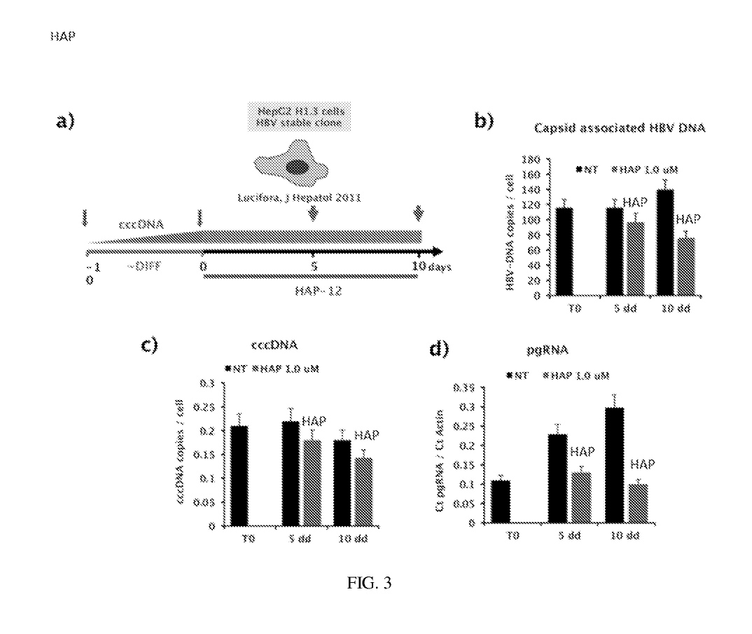

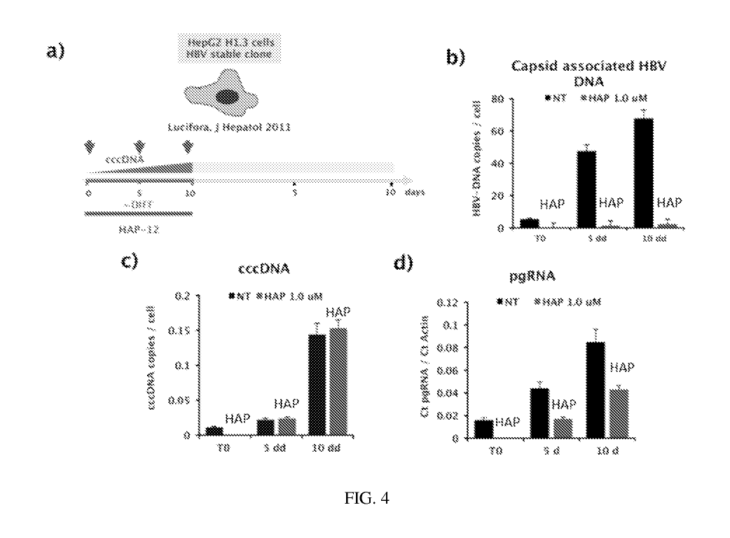

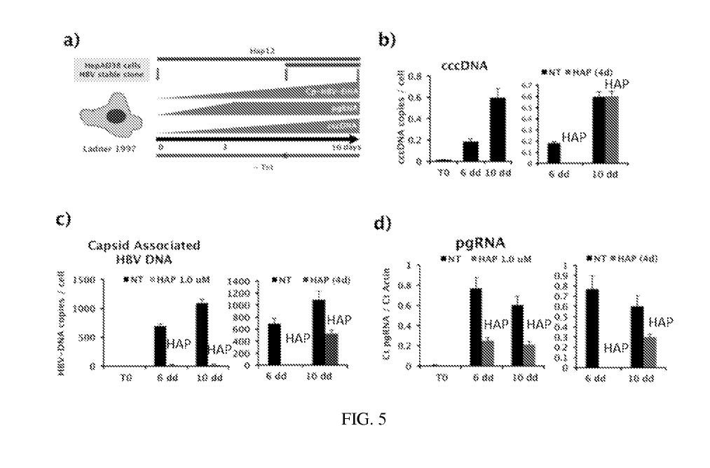

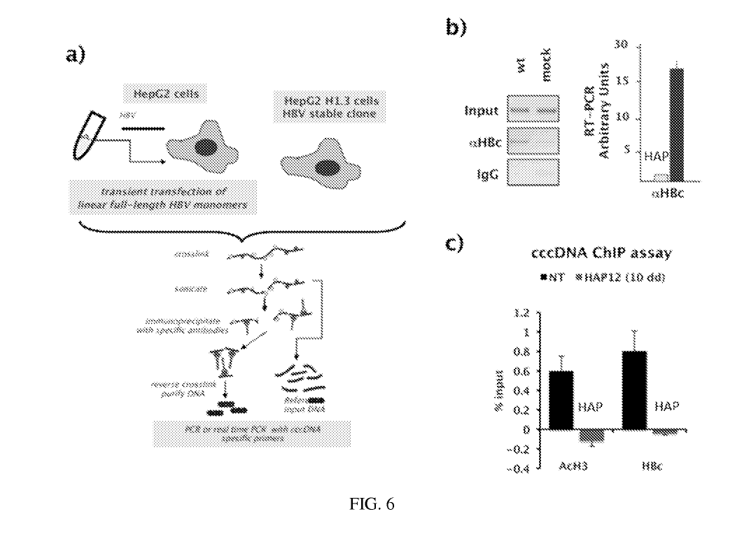

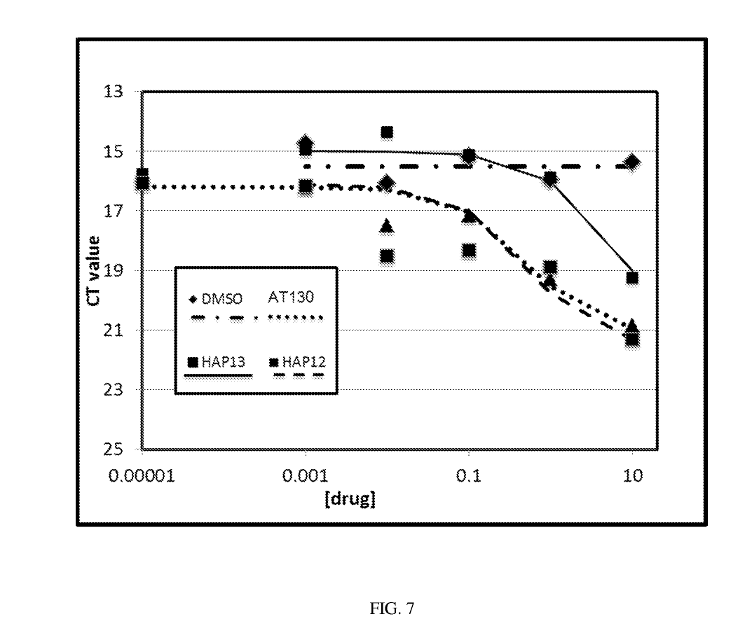

FIG. 3 HAP12 targets cccDNA transcription (2) In the HepG2 H1.3 stable cell line the HAP12 inhibitory effect on pgRNA transcription (d) and HBV replication (b) was confirmed. a) The HepG2 H1,3 HBV stable clone accumulates cccDNA when cultured in conditioned medium at high confluence. HAP12 treatment is started at day 10 of “differentiation” when HBV replication is high and the cccDNA pool is expanded. pgRNA is transcribed from both cccDNA and integrated HBV. b) Cytoplasmic HBV core particles, nuclear cccDNA (c) and total mRNAs (d) were isolated from untreated and HAP12-treated cells at the indicated time points. HBV-DNA, cccDNA and pgRNA results are expressed as in FIG. 2. Histograms show mean values from two independent experiments; bars indicate standard deviations (SD).

FIG. 4 HAP12 does not prevent cccDNA formation/accumulation. By treating the HepG2 H1.3 cells with HAP12 before the establishment of the cccDNA pool it was shown that cccDNA formation and accumulation are not targeted by HAP12. Under these conditions the effect on HBV replication is maximal. a) HAP12 treatment is started at day 0 of “differentiation” when HBV replication and cccDNA levels are very low. b) Cytoplasmic HBV core particles, nuclear cccDNA (c) and total mRNAs (d) were isolated from untreated and HAP12-treated cells at the indicated time points. HBV-DNA, cccDNA and pgRNA results are expressed as in FIG. 2. Histograms show mean values from two independent experiments; bars indicate standard deviations (SD).

FIG. 5 HAP12 targets cccDNA transcription in AD38 cells. HAP12 inhibitory effect on HBV replication (c) and pgRNA transcription (d) was further confirmed In the AD38 stable cell line. a) Upon tetracycline removal, the AD38 cells express pgRNA, accumulate subviral particles in the cytoplasm and secrete HBV virions in the cell supernatant (a). The cccDNA pool is built up from the recycling of mature core particles to the nucleus after HBV replication is started from the pgRNA initially transcribed from the “tet-regulated” HBV integrated DNA (b). HAP12 treatment is started at day 0 (left panels) or day 6 (right panels). c) Cytoplasmic HBV core particles, and total mRNAs (d) were isolated from untreated and HAP12-treated cells at the indicated time points. HBV DNA, cccDNA and pgRNA results are expressed as in FIG. 2. All histograms show mean values from two independent experiments; bars indicate standard deviations (SD).

FIG. 6 HAP12 interferes with HBc binding to the cccDNA. It is herein described that using a cccDNA ChIP assay (a), that HBc is recruited onto the cccDNA in HBV replicating HepG2 cells (b) and in the HepG2 H1.3 stable cell line. HAP12 treatment (10 days) strongly inhibited cccDNA HBc occupancy in HepG2 H1.3 cells and a sharp decrease in cccDNA-bound H3 histone acetylation. It is believe that this finding is in agreement with the observed inhibition of cccDNA transcription and pgRNA production in HAP12 treated cells. HAP12 treatment is started at day 10 of HepG2 H1.3 cells “differentiation” (see legend to FIG. 3). Cross-linked chromatin was prepared from untreated and HAP12-treated cells at TO (before treatment, 10 gg “differentiation”) and at T10 (10 days of exposure to HAP12, 20 gg from beginning of “differentiation”) and immune-precipitated with a relevant control IgG or anti-AcH4 antibody or anti-HBc antibody (USBiological, #H1905-15). ChIPped chromatin was analyzed by qPCR with HBV cccDNA selective primers. Results are expressed as % of input. Histograms show mean values from two independent experiments; bars indicate standard deviations (SD).

FIG. 7 Several compounds were used to treat a transient transfection of Huh-7 cells (Huh-7 is a well differentiated hepatocyte derived cellular carcinoma cell line). Cytoplasmic RNA was harvested and quantified by RT-per. Treatment/dynamic range/ED50: DMSO/1.6/NA; AT130/25/0.5 μM; HAP13/20/5 μM; HAP12/36/0.5 μM.

### DETAILED DESCRIPTION

Several illustrative embodiments of the invention are described by the following enumerated clauses:

1. A method for treating a patient having an infection by hepatitis B virus (HBV), the method comprising the step of administering to the patient a therapeutically effective amount a compound capable of inhibiting accumulation of HBV pgRNA in an HBV infected cell of the patient.

2. The method of clause 1 wherein the therapeutically effective amount is greater than or equal to that corresponding to an in vitro dose that is 10-fold greater than required to suppress DNA synthesis or 1 μM in a cell-based assay of pgRNA accumulation in HepG2 H1.3 cells.

3. The method of clause 1 or 2 wherein the compound has the formula

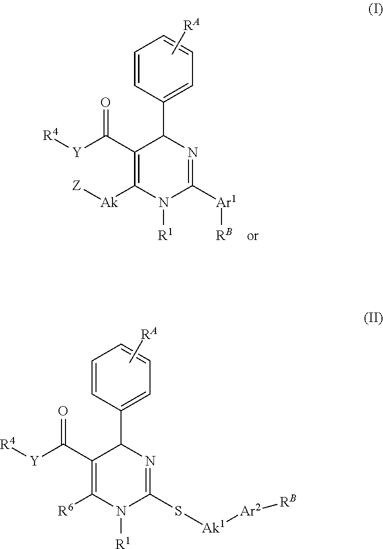

wherein

Ar¹ and Ar² are each independently selected from aryl or heteroaryl;

R¹ is hydrogen or a pro-drug forming group;

Ak is alkylene;

Ak¹ is (CH₂)n, where n is 1 to 4;

Z is hydrogen or

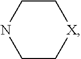

where X is CHN₃, C═O, C═NR⁵, —C(O)N(R^{N})—, or NR^{N}, where R⁵ is hydroxy or a derivative thereof or amino or a derivative thereof, and R^{N} is selected from the group consisting of alkyl, alkenyl, alkynyl, heteroalkyl, arylalkyl, heteroarylalkyl, alkyl-C(O), heteroalkyl-C(O), alkoxyl-C(O), alkynyl-C(O), alkylacylamino-C(O), and heteroalkylacylamino-C(O), each of which is optionally substituted;

R⁴ is alkyl, heteroalkyl, alkenyl, or alkynyl, each of which is optionally substituted;

Y is O, or HN;

R^{A} represents from 0 to 3 substituents independently selected in each instance from the group consisting of halo, and alkyl, heteroalkyl, aryl, heteroaryl, amino and derivatives thereof, and hydroxyl and derivatives thereof, each of which is optionally substituted; and

R^{B} represents from 0 to 3 substituents independently selected in each instance from the group consisting of halo, and alkyl, heteroalkyl, aryl, heteroaryl, amino and derivatives thereof, and hydroxyl and derivatives thereof, each of which is optionally substituted; and

R⁶ is in each instance independently selected from the group consisting of hydrogen and Ak-Z¹, where Ak is alkylene, and Z¹ is hydrogen or NR²R³; where R² and R³ are independently in each instance selected from the group consisting of hydrogen, and alkyl, cycloalkyl, heteroalkyl and heterocycloalkyl, each of which is optionally substituted, or

R² and R³ are taken together with the attached nitrogen to form

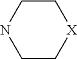

wherein X is CHN₃, C═O, —C(O)N(R^{Na})—, C═NR⁵, or NR^{Na}; where R⁵ is hydroxy or a derivative thereof or amino or a derivative thereof; and R^{Na} is selected from the group consisting of hydrogen, and alkyl, alkenyl, alkynyl, heteroalkyl, arylalkyl, heteroarylalkyl, alkyl-C(O), heteroalkyl-C(O), alkoxyl-C(O), alkynyl-C(O), alkylacylamino-C(O), and heteroalkylacylamino-C(O), each of which is optionally substituted.

4. A method for identifying a compound useful for the treatment of infection by hepatitis B virus (HBV), comprising:

contacting a cell infected with HBV with a test compound in a culture medium, or administering a potential compound to an animal;

retrieving a sample from the cell, the culture medium, or from tissue of the animal, at one or more time points;

analyzing the sample for one or more attributes selected from the group consisting of HBV cccDNA concentration, amount of methylated cccDNA, acetylation state of cccDNA, HBV cccDNA transcription, HBV RNA concentration in cellular cytoplasm, HBV RNA concentration in the cell nucleus, concentration of unassembled capsid protein, and HBV S antigen concentration; and

identifying the compound as useful for treating hepatitis B based on the reduction or increase of one of more of the attributes.

5. The method of clause 4, wherein the analyzing step comprises analyzing HBV capsid stabilization or capsid nucleation.

6. The method of clause 4 or 5, wherein the HBV RNA concentration is selected from pgRNA, subgeneric subgenomic RNA, or spliced RNA.

7. The method of any one of clauses 4 to 6 wherein the analyzing step comprises determining the effect the compound on capsid stability, the effect of the compound on nucleation of assembly, the affinity of the compound for capsid, the affinity of the compound for dimer, or the ability of the compound to induce allosteric effect.

8. The method of any one of clauses 4 to 7, further comprising varying the concentration of the potential compound until one or more of the attributes is reduced or increased.

9. The method of any one of clauses 4 to 8, wherein the candidate compound is heteroaryldihydropyrimidine compound.

10. The method any one of clauses 4 to 8, wherein the candidate compound is the compound described in clause 3.

11 The method of clause 8, wherein the concentration of the potential compound is about 1 μM or higher.

12. The method of any one of clauses 4 to 11, wherein the tissue is liver tissue.

13. The method of any one of clauses 4 to 12, wherein the animal is a rodent or human.

14. The method of any one of clauses 4 to 13, wherein the cell is from a cell line derived from human hepatocytes (e.g. Huh7, AD38, HepG2, or HepG2.2.15).

15. A method of reducing pgRNA transcription in a HBV infected cell, comprising contacting the cell with a heteroaryldihydropyrimidine compound.

16. A method of reducing pgRNA transcription in a HBV infected cell, comprising contacting the cell with the compound described in clause 3.

17. The method of clause 3, 4, 15, or 16 wherein R^{A} represents 2-chloro-4-fluoro.

18. The method of clause 3, 4, 15, or 16 wherein Ar¹ is 2-pyridyl.

19. The method of clause 3, 4, 15, or 16 wherein R^{B} represents 0 substituents.

20. The method of clause 3, 4, 15, or 16 wherein Y is O.

21. The method of clause 3, 4, 15, or 16 wherein R⁴ is methyl.

22. The method of clause 3, 4, 15, or 16 where in Ak is methylene.

23. The method of clause 3, 4, 15, or 16 wherein Z is hydrogen.

24. The method of clause 3, 4, 15, or 16 wherein Z-Ak is CH₃.

25. The method of clause 22 wherein X is C═O, —C(O)N(R^{N})—, or NR^{N}.

26. The method of clause 25 wherein X is C═O.

27. The method of clause 3, 4, 15, or 16 wherein R⁶ is methyl.

28. The method of clause 3, 4, 15, or 16 wherein R⁶ is

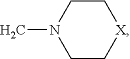

where X is C═O, —C(O)N(R^{Na})—, or NR^{Na}, where R^{Na} is hydrogen or alkyl, alkenyl, alkynyl, heteroalkyl, arylalkyl, heteroarylalkyl, alkyl-C(O), heteroalkyl-C(O), alkylacylamino-C(O), and heteroalkylacylamino-C(O), each of which is optionally substituted.

29. The method of clause 28 wherein X is C═O.

104. A method for identifying a compound useful for the treatment of infection by hepatitis B virus (HBV), comprising:

contacting a cell infected with HBV with the compound in a culture medium, or administering a potential compound to an animal;

retrieving a sample from the cell, the culture medium, or from tissue of the animal, at one or more time points;

analyzing the sample for one or more attributes selected from the group consisting of HBV cccDNA concentration, amount of methylated cccDNA, acetylation state of cccDNA, HBV cccDNA transcription, HBV RNA concentration in cellular cytoplasm, HBV RNA concentration in the cell nucleus, concentration of unassembled capsid protein, HBV capsid stabilization, HBV capsid nucleation, and HBV S antigen concentration; and

identifying the compound as useful for treating hepatitis B based on the reduction or increase of one of more of the attributes.

105. The method of clause 104, wherein the analyzing step comprises analyzing HBV capsid stabilization or capsid nucleation.

106. The method of clause 104 or 105, wherein the HBV RNA concentration is selected from pgRNA, subgeneric subgenomic RNA, or spliced RNA.

107. The method of clause 104 or 105 wherein the analyzing step comprises determining the effect the compound on capsid stability, the effect of the compound on nucleation of assembly, the affinity of the compound for capsid, the affinity of the compound for Cp dimer, or the ability of the compound to induce an allosteric effect.

108. The method of any one of clauses 104 to 107, further comprising varying the concentration of the potential compound until one or more of the attributes is reduced or increased.

109. The method of any one of clauses 104 to 108, wherein the compound is heteroaryldihydropyrimidine compound.

110. The method of any one of clauses 104 to 108, wherein the compound is the compound described in clause 3.

111. The method of claim 108, wherein the concentration of the compound is from about 0.1 μM to about 1 μM or from about 1 μM to about 10 μM or from about 10 μM to about 50 μM.

112. The method of any one of clauses 104 to 111, wherein the tissue is liver tissue.

113. The method of any one of clauses 104 to 112, wherein the animal is a rodent or a human.

114. The method of any one of clauses 104 to 113, wherein the cell is from a cell line derived from human hepatocytes (e.g. Huh7, AD38, HepG2, or HepG2.2.15).

115. A method of reducing pgRNA transcription in a HBV infected cell, comprising contacting the cell with a heteroaryldihydropyrimidine compound.

116. A method of reducing pgRNA transcription in a HBV infected cell, comprising contacting the cell with the compound described in clause 3.

117. The method of clause 3, 110, or 116 wherein R^{A} represents 2-cholor-4-fluoro.

118. The method of clause 3, 110, or 116 wherein Ar¹ is 2-pyridyl.

119. The method of clause 3, 110, or 116 wherein R^{B} represents 0 substituents.

120. The method of clause 3, 110, or 116 wherein Y is O.

121. The method of clause 3, 110, or 116 wherein R⁴ is methyl.

122. The method of clause 3, 110, or 116 where in Ak is methylene.

123. The method of clause 3, 110, or 116 wherein Z is hydrogen.

124. The method of clause 3, 110, or 116 wherein Z-Ak is CH₃.

125. The method of clause 122 wherein X is C═O, —C(O)N(R^{N})—, or NR^{N}.

126. The method of clause 125 wherein X is C═O.

127. The method of clause 3, 110, or 116 wherein R⁶ is methyl.

128. The method of clause 3, 110, or 116 wherein R⁶ is

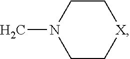

where X is C═O, —C(O)N(R^{Na})—, or NR^{Na}, where R^{Na} is hydrogen or alkyl, alkenyl, alkynyl, heteroalkyl, arylalkyl, heteroarylalkyl, alkyl-C(O), heteroalkyl-C(O), alkylacylamino-C(O), and heteroalkylacylamino-C(O), each of which is optionally substituted.

129. The method of clause 128 wherein X is C═O.

130. The method of any one of the preceding clauses wherein pgRNA transcription is reduced by a factor of from about 1.5 to about 2.5.

131. The method of any one of the preceding clauses wherein capsid associated HBV DNA is reduced by a factor of from about 2 to about 50.

132. The method of any one of the proceding clauses wherein the cccDNA is reduced by from about 25% to about 50%.

133. The method of any one of the preceding clauses wherein the compound is not HAP1, HAP12, or HAP13.

In addition, various genera and subgenera of each of R¹, R², R³, R⁴, R⁵, R⁶, Ar¹, Ar², X, Ak, Ak¹, R^{A}, R^{B}, and Z are described herein. It is to be understood that all possible combinations of the various genera and subgenera of each of R¹, R², R³, R⁴, R⁵, R⁶, Ar¹, Ar², X, Ak, Ak¹, R^{A}, R^{B}, and Z described herein represent additional illustrative embodiments of compounds of the invention described herein. It is to be further understood that each of those additional illustrative embodiments of compounds may be used in any of the compositions, methods, and/or uses described herein.

In another embodiment, pharmaceutical compositions containing one or more of the compounds described herein are also described. In one aspect, the compositions include a therapeutically effective amount of the one or more compounds for treating a patient with hepatitis B. It is to be understood that the compositions may include other component and/or ingredients, including, but not limited to, other therapeutically active compounds, and/or one or more carriers, diluents, excipients, and the like. In another embodiment, methods for using the compounds and pharmaceutical compositions for treating patients with hepatitis B are also described herein. In one aspect, the methods include the step of administering one or more of the compounds and/or compositions described herein to a patient with hepatitis B. In another aspect, the methods include administering a therapeutically effective amount of the one or more compounds and/or compositions described herein for treating patients with hepatitis B. In another embodiment, uses of the compounds and compositions in the manufacture of a medicament for treating patients with hepatitis B are also described herein. In one aspect, the medicaments include a therapeutically effective amount of the one or more compounds and/or compositions for treating a patient with hepatitis B.

It is appreciated herein that the compounds described herein may be used alone or in combination with other compounds useful for treating hepatitis B in the methods described herein, including those compounds that may be therapeutically effective by the same or different modes of action. In addition, it is appreciated herein that the compounds described herein may be used in combination with other compounds that are administered to treat other symptoms of hepatitis B.

In each of the foregoing and following embodiments, it is to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non crystalline and/or amorphous forms of the compounds.

In another embodiment, use of the following illustrative compounds in any of the methods described herein is described.

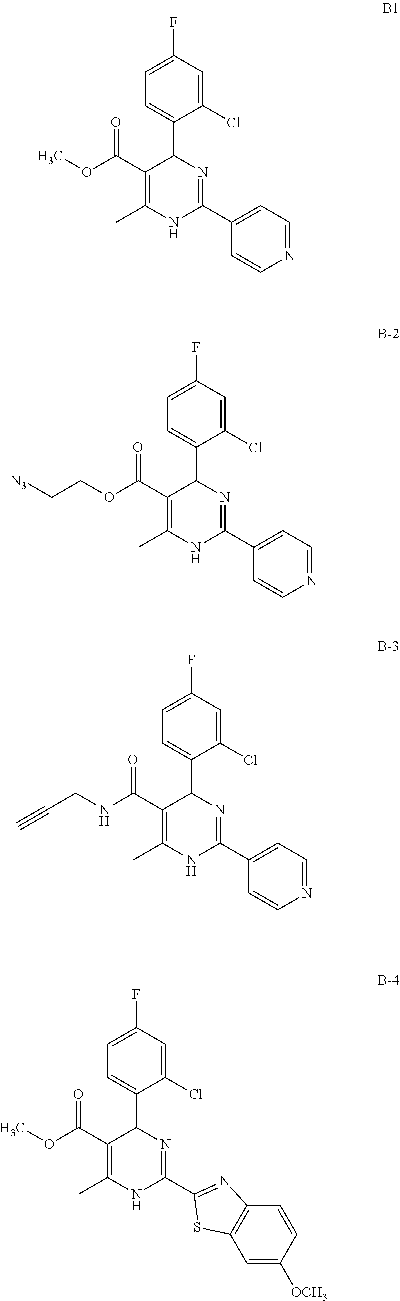 [SMILES: COC(=O)C1=C(C)NC(c2ccncc2)=NC1c1ccc(F)cc1Cl ; CC1=C(C(=O)OCCN=[N+]=[N-])C(c2ccc(F)cc2Cl)N=C(c2ccncc2)N1 ; C#CCNC(=O)C1=C(C)NC(c2ccncc2)=NC1c1ccc(F)cc1Cl ; COC(=O)C1=C(C)NC(c2nc3ccc(OC)cc3s2)=NC1c1ccc(F)cc1Cl]

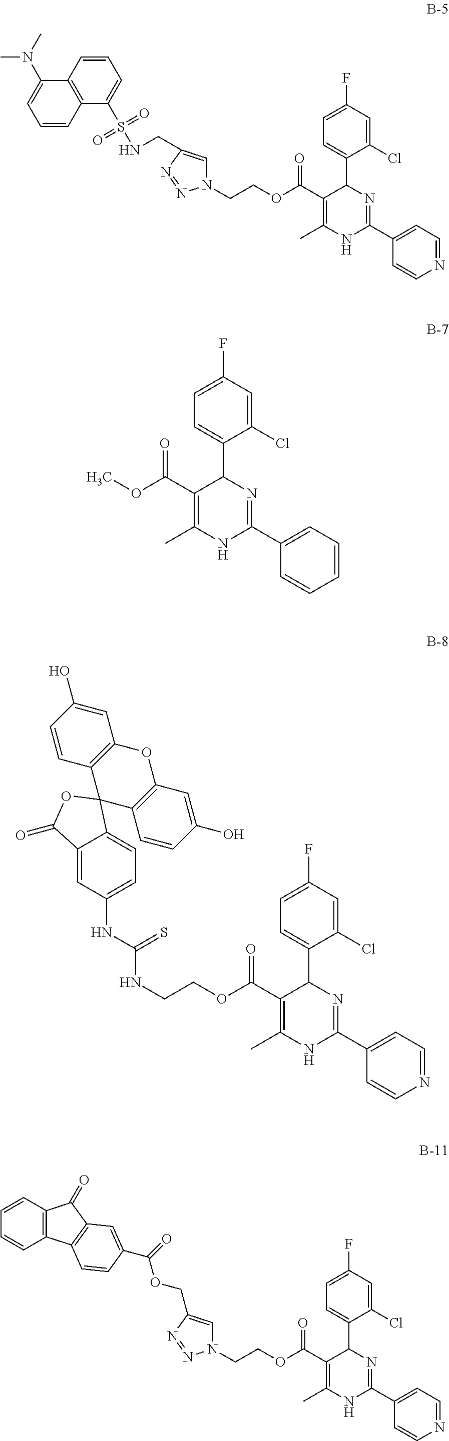 [SMILES: CC1=C(C(=O)OCCn2cc(CNS(=O)(=O)c3cccc4c(N(C)C)cccc34)nn2)C(c2ccc(F)cc2Cl)N=C(c2ccncc2)N1 ; COC(=O)C1=C(C)NC(c2ccccc2)=NC1c1ccc(F)cc1Cl ; CC1=C(C(=O)OCCNC(=S)Nc2ccc3c(c2)C(=O)OC32c3ccc(O)cc3Oc3cc(O)ccc32)C(c2ccc(F)cc2Cl)N=C(c2ccncc2)N1 ; CC1=C(C(=O)OCCn2cc(COC(=O)c3ccc4c(c3)C(=O)c3ccccc3-4)nn2)C(c2ccc(F)cc2Cl)N=C(c2ccncc2)N1]

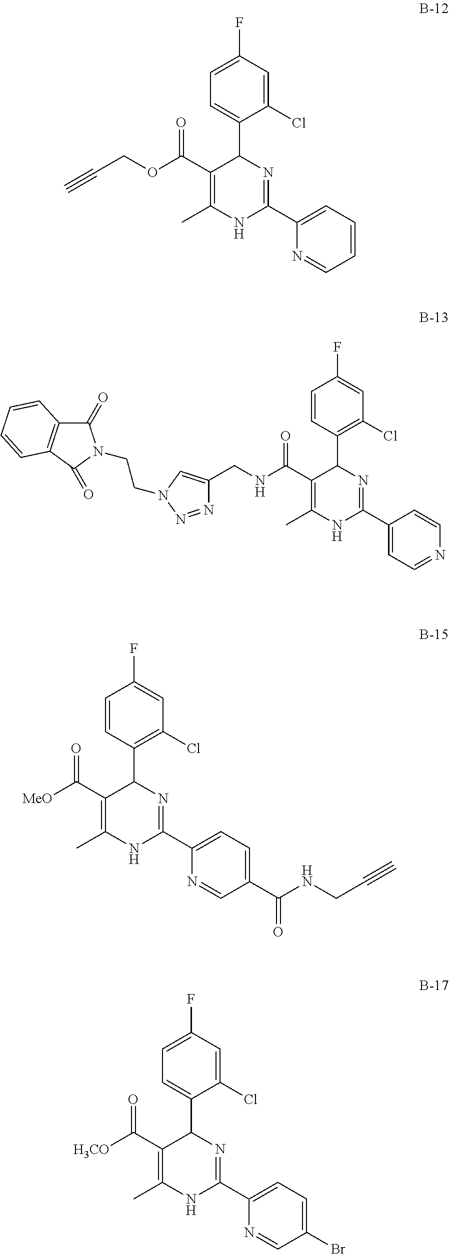 [SMILES: C#CCOC(=O)C1=C(C)NC(c2ccccn2)=NC1c1ccc(F)cc1Cl ; CC1=C(C(=O)NCc2cn(CCN3C(=O)c4ccccc4C3=O)nn2)C(c2ccc(F)cc2Cl)N=C(c2ccncc2)N1 ; C#CCNC(=O)c1ccc(C2=NC(c3ccc(F)cc3Cl)C(C(=O)OC)=C(C)N2)nc1 ; COC(=O)C1=C(C)NC(c2ccc(Br)cn2)=NC1c1ccc(F)cc1Cl]

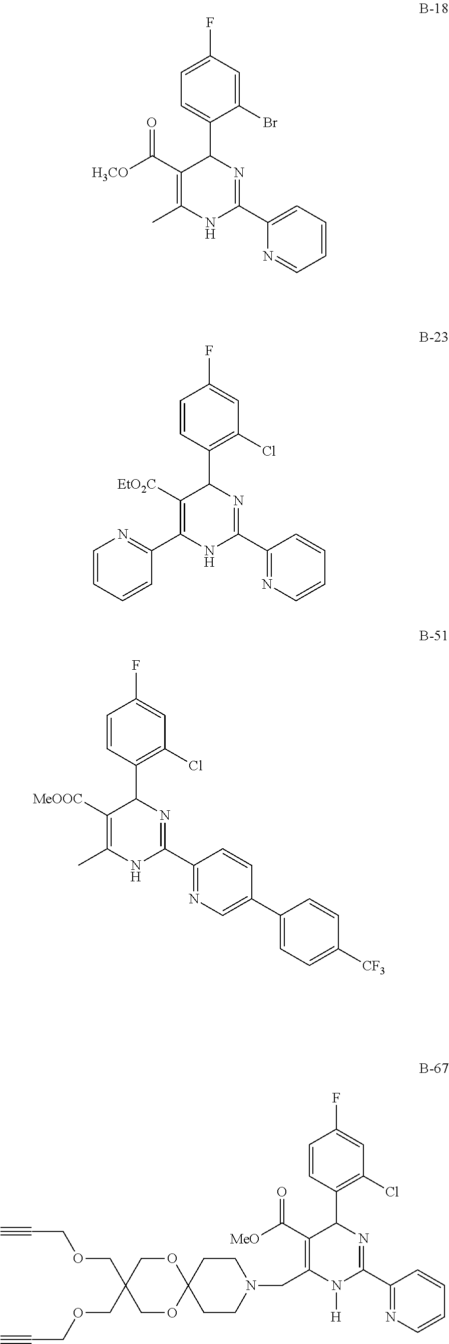 [SMILES: COC(=O)C1=C(C)NC(c2ccccn2)=NC1c1ccc(F)cc1Br ; CCOC(=O)C1=C(c2ccccn2)NC(c2ccccn2)=NC1c1ccc(F)cc1Cl ; COC(=O)C1=C(C)NC(c2ccc(-c3ccc(C(F)(F)F)cc3)cn2)=NC1c1ccc(F)cc1Cl ; C#CCOCC1(COCC#C)COC2(CCN(CC3=C(C(=O)OC)C(c4ccc(F)cc4Cl)N=C(c4ccccn4)N3)CC2)OC1]

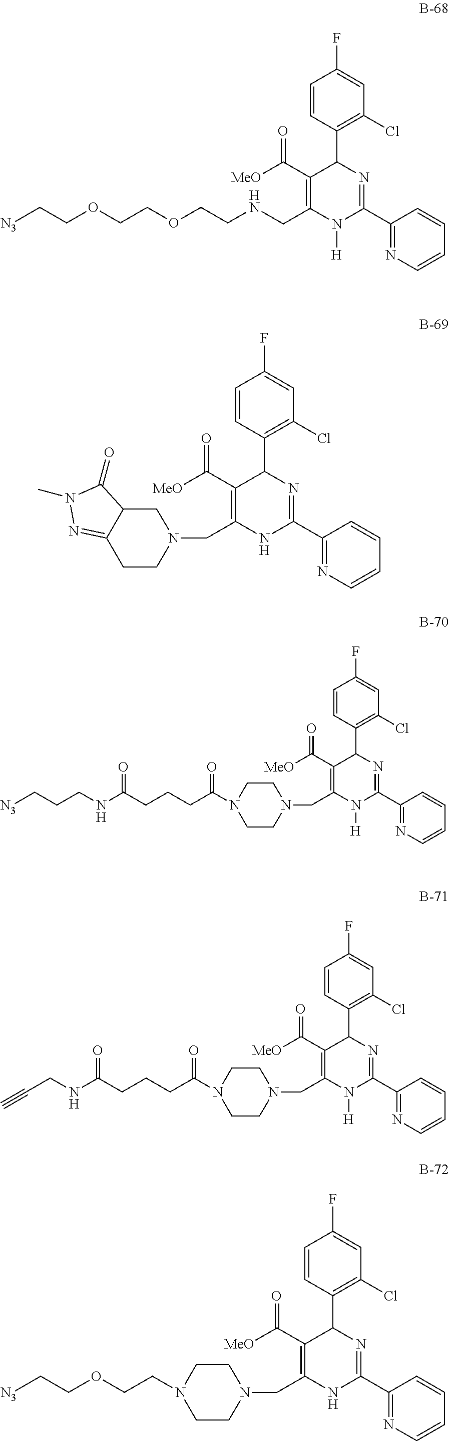 [SMILES: COC(=O)C1=C(CNCCOCCOCCN=[N+]=[N-])NC(c2ccccn2)=NC1c1ccc(F)cc1Cl ; COC(=O)C1=C(CN2CCC3=NN(C)C(=O)C3C2)NC(c2ccccn2)=NC1c1ccc(F)cc1Cl ; COC(=O)C1=C(CN2CCN(C(=O)CCCC(=O)NCCCN=[N+]=[N-])CC2)NC(c2ccccn2)=NC1c1ccc(F)cc1Cl ; C#CCNC(=O)CCCC(=O)N1CCN(CC2=C(C(=O)OC)C(c3ccc(F)cc3Cl)N=C(c3ccccn3)N2)CC1 ; COC(=O)C1=C(CN2CCN(CCOCCN=[N+]=[N-])CC2)NC(c2ccccn2)=NC1c1ccc(F)cc1Cl]

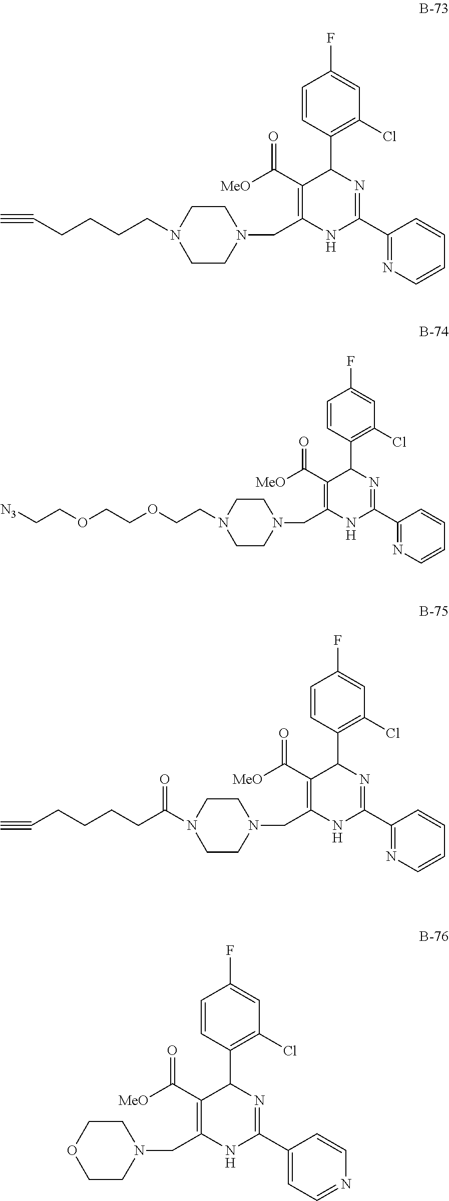 [SMILES: C#CCCCCN1CCN(CC2=C(C(=O)OC)C(c3ccc(F)cc3Cl)N=C(c3ccccn3)N2)CC1 ; COC(=O)C1=C(CN2CCN(CCOCCOCCN=[N+]=[N-])CC2)NC(c2ccccn2)=NC1c1ccc(F)cc1Cl ; C#CCCCCC(=O)N1CCN(CC2=C(C(=O)OC)C(c3ccc(F)cc3Cl)N=C(c3ccccn3)N2)CC1 ; COC(=O)C1=C(CN2CCOCC2)NC(c2ccncc2)=NC1c1ccc(F)cc1Cl]

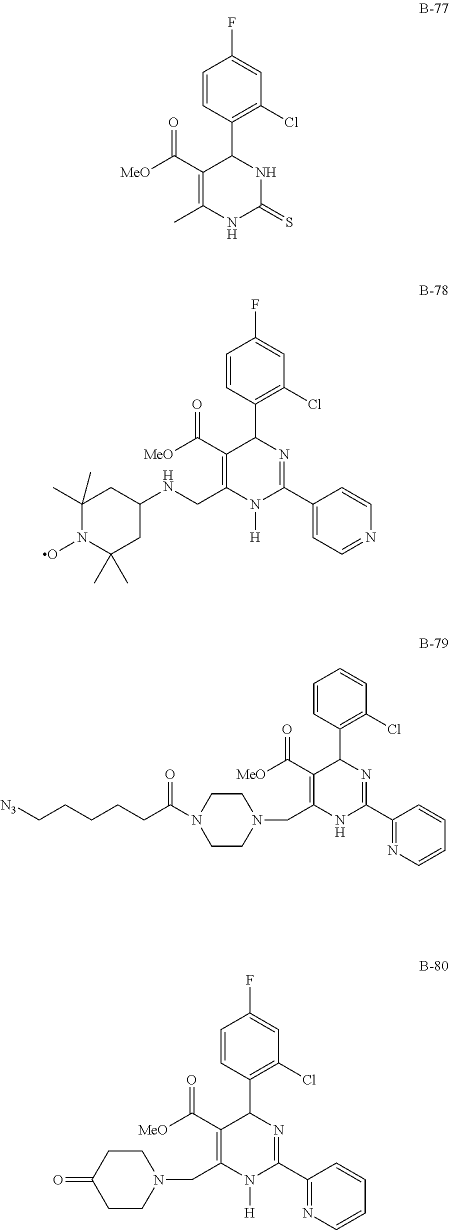

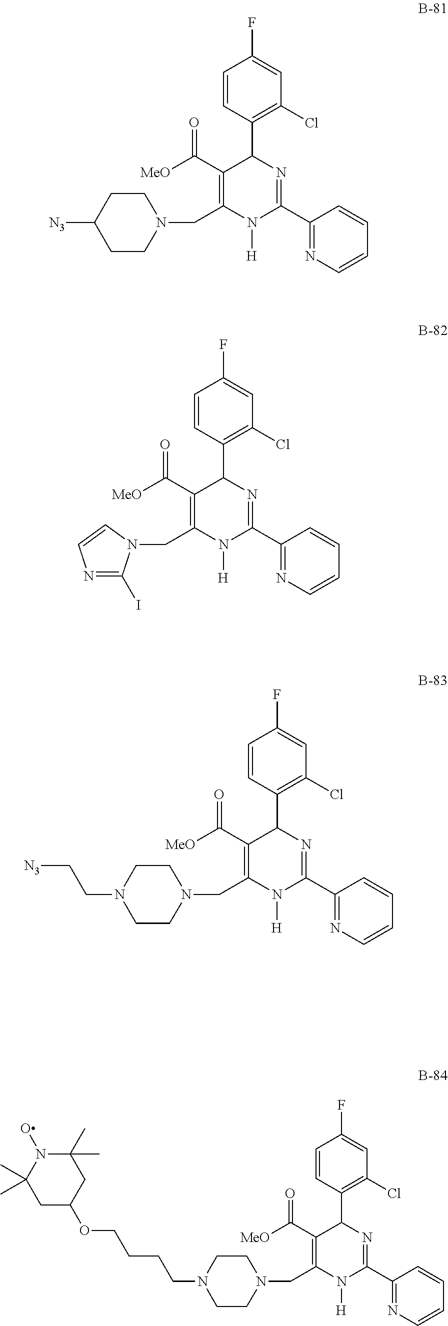

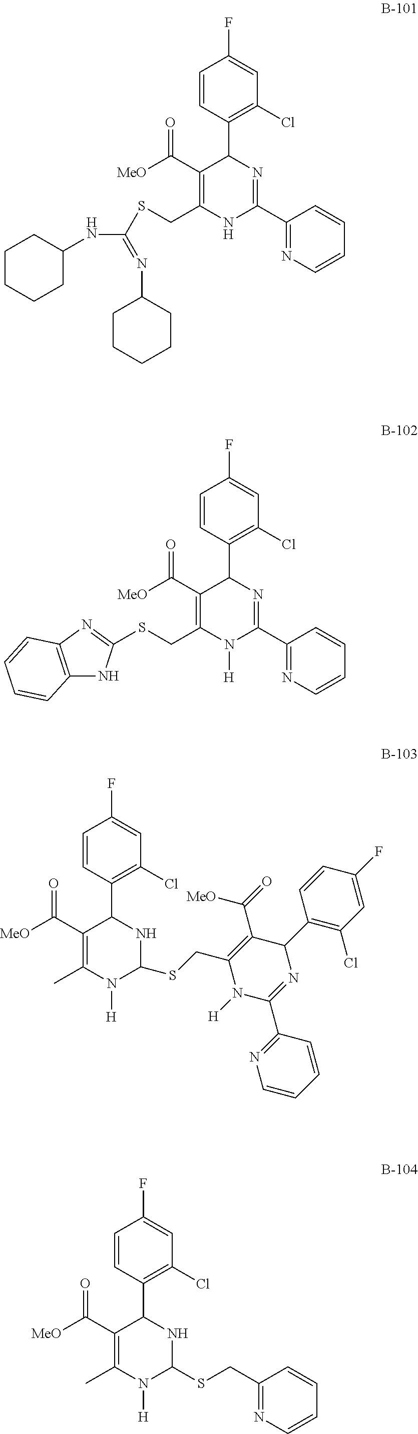 [SMILES: COC(=O)C1=C(CS/C(=N/C2CCCCC2)NC2CCCCC2)NC(c2ccccn2)=NC1c1ccc(F)cc1Cl ; COC(=O)C1=C(CSc2nc3ccccc3[nH]2)NC(c2ccccn2)=NC1c1ccc(F)cc1Cl ; COC(=O)C1=C(CSC2NC(C)=C(C(=O)OC)C(c3ccc(F)cc3Cl)N2)NC(c2ccccn2)=NC1c1ccc(F)cc1Cl ; COC(=O)C1=C(C)NC(SCc2ccccn2)NC1c1ccc(F)cc1Cl]

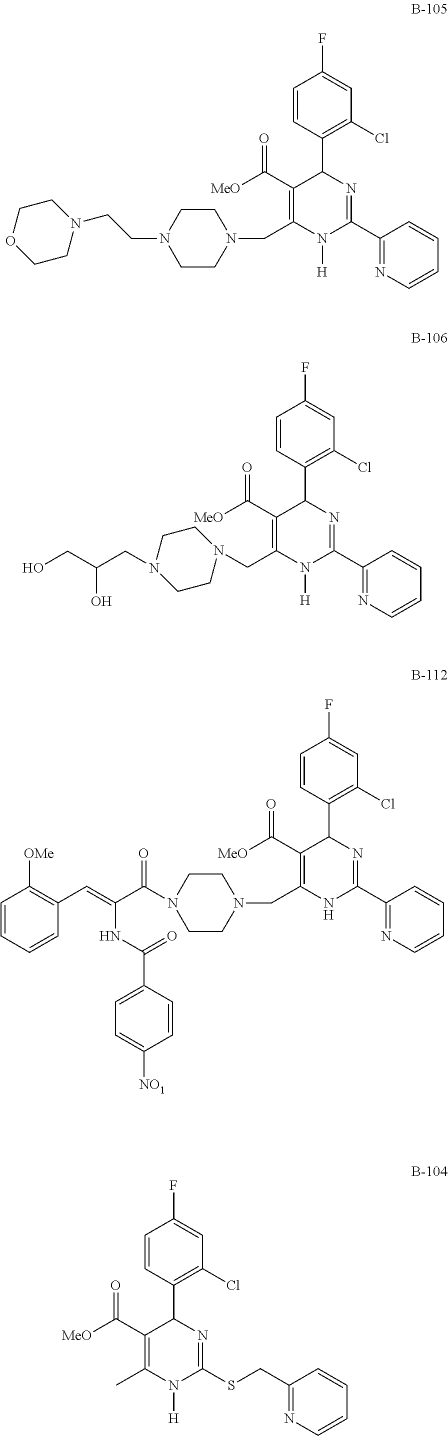

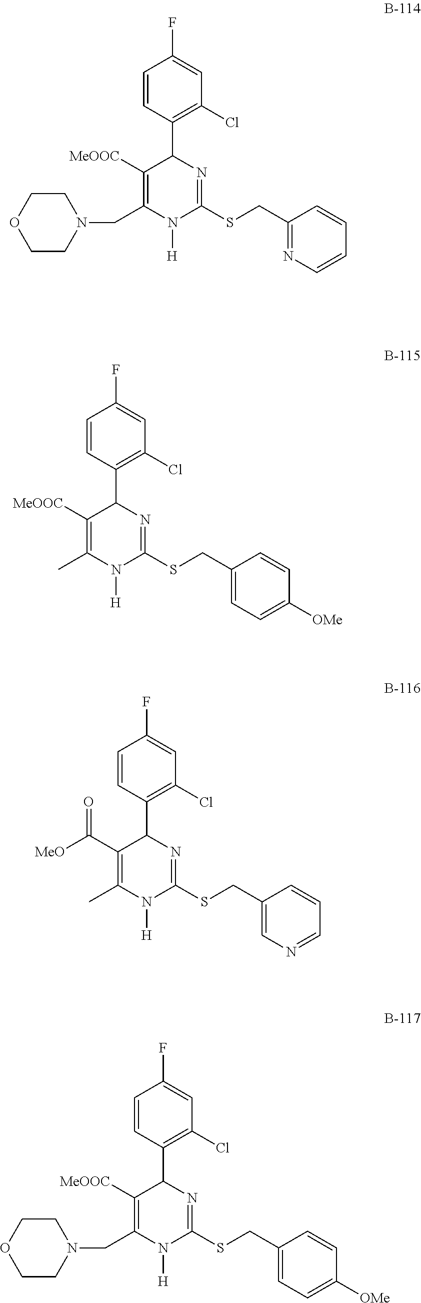 [SMILES: COC(=O)C1=C(CN2CCOCC2)NC(SCc2ccccn2)=NC1c1ccc(F)cc1Cl ; COC(=O)C1=C(C)NC(SCc2ccc(OC)cc2)=NC1c1ccc(F)cc1Cl ; COC(=O)C1=C(C)NC(SCc2cccnc2)=NC1c1ccc(F)cc1Cl ; COC(=O)C1=C(CN2CCOCC2)NC(SCc2ccc(OC)cc2)=NC1c1ccc(F)cc1Cl]

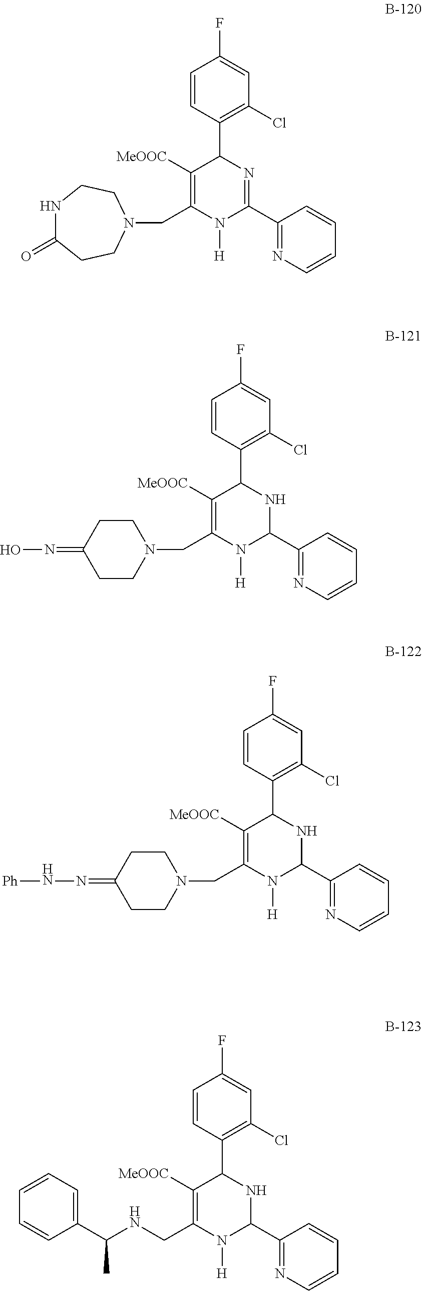 [SMILES: COC(=O)C1=C(CN2CCNC(=O)CC2)NC(c2ccccn2)=NC1c1ccc(F)cc1Cl ; COC(=O)C1=C(CN2CCC(=NO)CC2)NC(c2ccccn2)NC1c1ccc(F)cc1Cl ; COC(=O)C1=C(CN2CCC(=NNc3ccccc3)CC2)NC(c2ccccn2)NC1c1ccc(F)cc1Cl ; COC(=O)C1=C(CN[C@@H](C)c2ccccc2)NC(c2ccccn2)NC1c1ccc(F)cc1Cl]

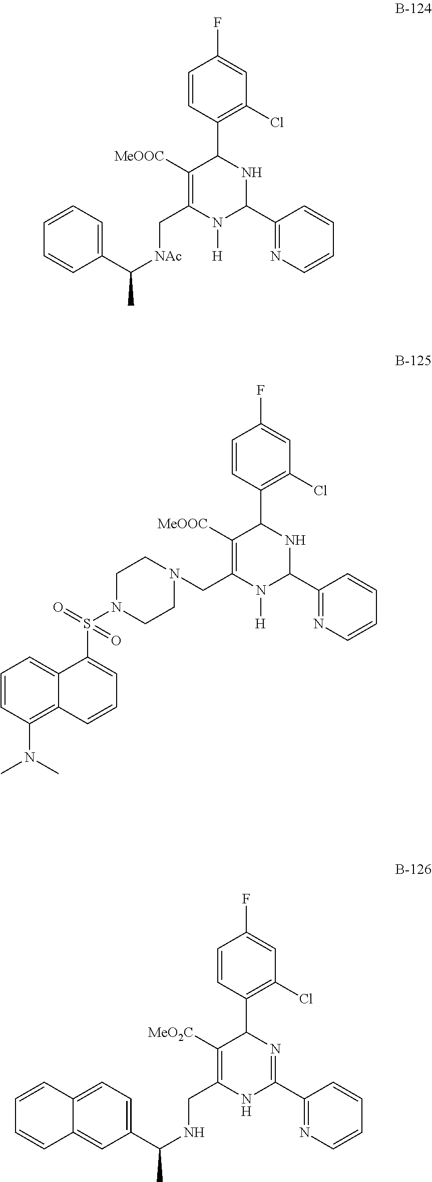

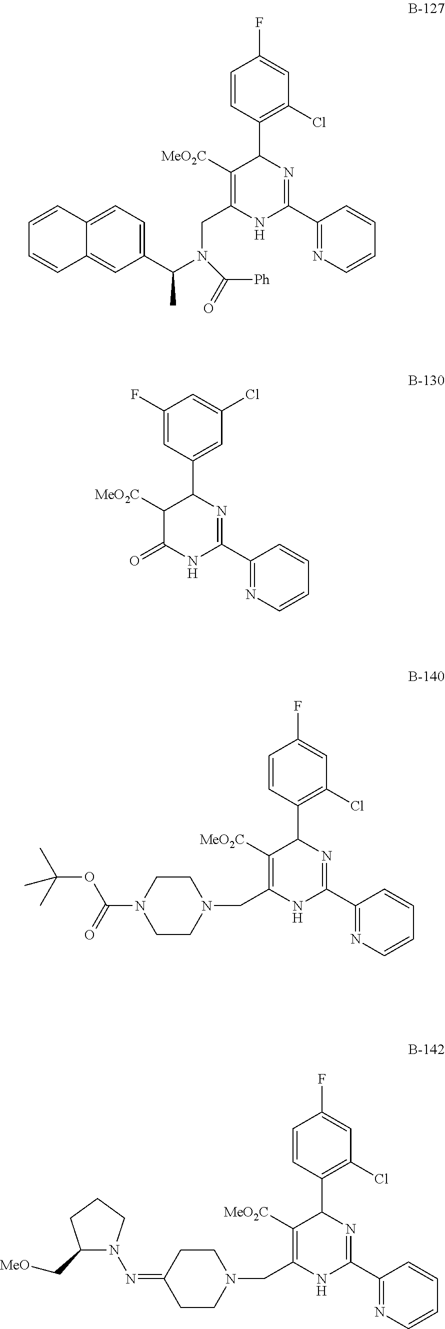 [SMILES: COC(=O)C1=C(CN(C(=O)c2ccccc2)[C@@H](C)c2ccc3ccccc3c2)NC(c2ccccn2)=NC1c1ccc(F)cc1Cl ; COC(=O)C1C(=O)NC(c2ccccn2)=NC1c1cc(F)cc(Cl)c1 ; COC(=O)C1=C(CN2CCN(C(=O)OC(C)(C)C)CC2)NC(c2ccccn2)=NC1c1ccc(F)cc1Cl ; COC[C@H]1CCCN1N=C1CCN(CC2=C(C(=O)OC)C(c3ccc(F)cc3Cl)N=C(c3ccccn3)N2)CC1]

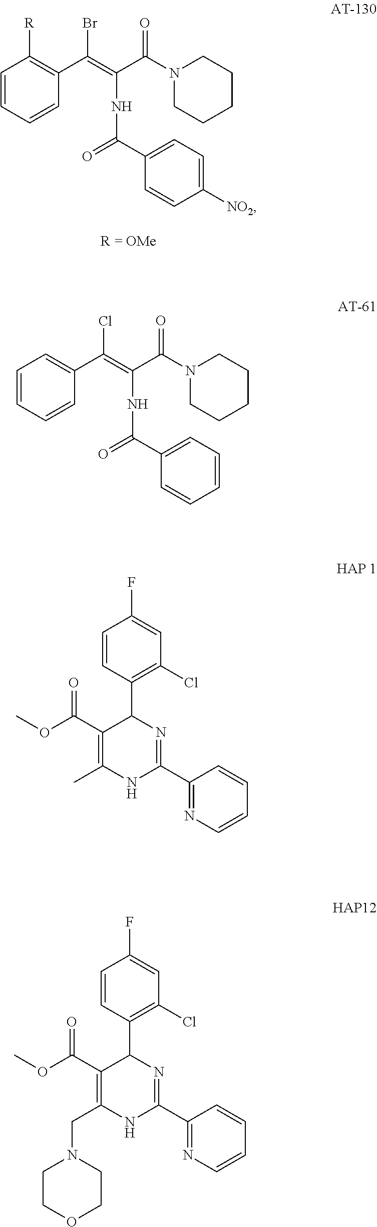

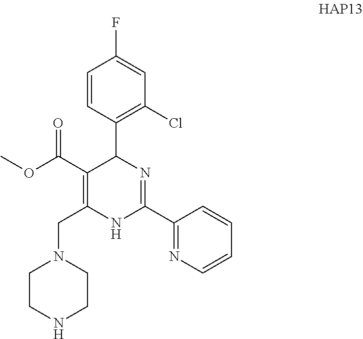 [SMILES: COC(=O)C1=C(CN2CCNCC2)NC(c2ccccn2)=NC1c1ccc(F)cc1Cl]

Illustrative derivatives include, but are not limited to, both those compounds that may be synthetically prepared from the compounds described herein, as well as those compounds that may be prepared in a similar way as those described herein, but differing in the selection of starting materials. In addition, it is to be understood that derivatives of those compounds also include the compounds having those same or different functional groups at different positions on the aromatic ring. Similarly, derivatives include parallel variations of other functional groups on the compounds described herein, such as R^{A}, R^{B}, and the like.

It is to be understood that such derivatives may include prodrugs of the compounds described herein, compounds described herein that include one or more protection or protecting groups, including compounds that are used in the preparation of other compounds described herein.

It is to be understood that each of the foregoing embodiments may be combined in chemically relevant ways to generate subsets of the embodiments described herein. Accordingly, it is to be further understood that all such subsets are also illustrative embodiments of the invention described herein

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be understood that in certain embodiments, each of the forgoing may be univalent (i.e. attached to the remainder of the formula via one attachment) or multivalent (i.e. attached to the remainder of the formula via more than one attachment). It is to be further understood that in certain embodiments, alkyl is advantageously of limited length, including C₁-C₂₄, C₁-C₁₂, C₁-C₈, C₁-C₆, and C₁-C₄. It is to be further understood that in certain embodiments alkenyl and/or alkynyl may each be advantageously of limited length, including C₂-C₂₄, C₂-C₁₂, C₂-C₈, C₂-C₆, and C₂-C₄. It is appreciated herein that shorter alkyl, alkenyl, and/or alkynyl groups may add less lipophilicity to the compound and accordingly will have different pharmacokinetic behavior. Illustrative alkyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl and the like.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄. Illustratively, such particularly limited length alkylene groups, including C₁-C₈, C₁-C₆, and C₁-C₄ may be referred to as lower alkylene. It is to be further understood that in certain embodiments alkenylene and/or alkynylene may each be advantageously of limited length, including C₂-C₂₄, C₂-C₁₂, C₂-C₈, C₂-C₆, and C₂-C₄. Illustratively, such particularly limited length alkenylene and/or alkynylene groups, including C₂-C₈, C₂-C₆, and C₂-C₄ may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain is cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. It is to be understood that in certain embodiments, each of the forgoing may be univalent (i.e. attached to the remainder of the formula via one attachment) or multivalent (i.e. attached to the remainder of the formula via more than one attachment). Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. It is to be understood that in certain embodiments, each of the forgoing may be univalent (i.e. attached to the remainder of the formula via one attachment) or multivalent (i.e. attached to the remainder of the formula via more than one attachment). In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylate and derivatives thereof” includes the group CO₂H and salts thereof, and esters and amides thereof, and CN.

As used herein, the term “sulfinyl or a derivative thereof” includes SO₂H and salts thereof, and esters and amides thereof.

As used herein, the term “sulfonyl or a derivative thereof” includes SO₃H and salts thereof, and esters and amides thereof.

As used herein, the term “phosphinyl or a derivative thereof” includes P(R)O₂H and salts thereof, and esters and amides thereof, where R is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted.

As used herein, the term “phosphonyl or a derivative thereof” includes PO₃H₂ and salts thereof, and esters and amides thereof.

As used herein, the term “hydroxylamino and derivatives thereof” includes NHOH, and alkyloxylNH alkenyloxylNH alkynyloxylNH heteroalkyloxylNH heteroalkenyloxylNH heteroalkynyloxylNH cycloalkyloxylNH cycloalkenyloxylNH cycloheteroalkyloxylNH cycloheteroalkenyloxylNH aryloxylNH arylalkyloxylNH arylalkenyloxylNH arylalkynyloxylNH heteroaryloxylNH heteroarylalkyloxylNH heteroarylalkenyloxylNH heteroarylalkynyloxylNH acyloxy, and the like, each of which is optionally substituted.

As used herein, the term “hydrazino and derivatives thereof” includes alkylNHNH, alkenylNHNH, alkynylNHNH, heteroalkylNHNH, heteroalkenylNHNH, heteroalkynylNHNH, cycloalkylNHNH, cycloalkenylNHNH, cycloheteroalkylNHNH, cycloheteroalkenylNHNH, arylNHNH, arylalkylNHNH, arylalkenylNHNH, arylalkynylNHNH, heteroarylNHNH, heteroarylalkylNHNH, heteroarylalkenylNHNH, heteroarylalkynylNHNH, acylNHNH, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, azido, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, azido, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)_{q}Z^{X}, where q is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, azido, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165; and Pathalk et al., Enzymic protecting group techniques in organic synthesis, Stereosel. Biocatal. 775-797 (2000). It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Illustrative esters, also referred to as active esters, include but are not limited to 1-indanyl, N-oxysuccinimide; acyloxyalkyl groups such as acetoxymethyl, pivaloyloxymethyl, β-acetoxyethyl, β-pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, α-ethoxycarbonyloxyethyl, β-ethoxycarbonyloxyethyl, and the like; dialkylaminoalkyl groups, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl and optionally substituted heteroaryl(C₂-C₁₆)alkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

It is to be understood that the embodiments described herein may be combined in all possible chemically relevant ways.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a co-therapy.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

As used herein, the term “treatment” or “treating” means any administration of a compound or composition described and includes (1) inhibiting the disease in a patient that is experiencing or displaying the pathology or symptomatology of infection by HBV (i.e., arresting further development of the pathology and/or symptomatology), (2) ameliorating the disease in a patient that is experiencing or displaying the pathology or symptomatology of infection by HBV (i.e., reversing or lessening the pathology and/or symptomatology), inhibiting or (4) preventing of chronic infection by HBV. The term “controlling” includes preventing, treating, eradicating, ameliorating or otherwise reducing the severity of the infection by HBV.

The term “administering” as used herein includes all means of introducing the compounds and compositions described herein to the patient, including, but are not limited to, oral (po), intravenous (iv), intramuscular (im), subcutaneous (sc), transdermal, inhalation, and the like. The compounds and compositions described herein may be administered in unit dosage forms and/or formulations containing conventional nontoxic pharmaceutically-acceptable carriers, adjuvants, and vehicles.

Illustrative routes of oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

### Examples

### Synthesis of a Representative HAP Compound

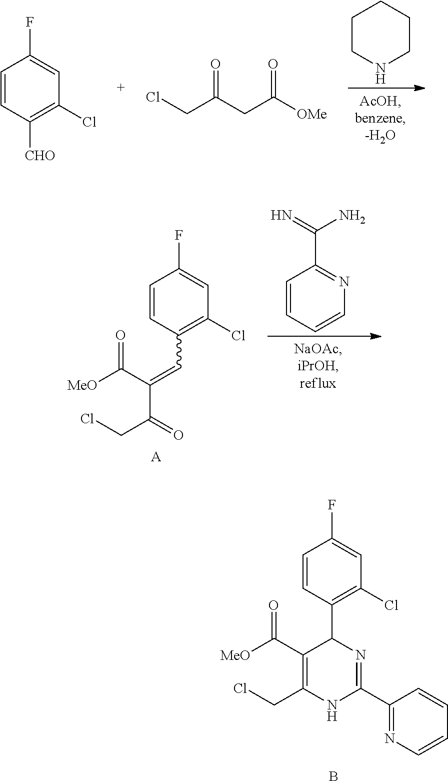

Compound A. A solution of methyl 4-chloroacetoacetate (2.43 mL, 20.0 mmol) and 2-chloro-4-fluorobenzaldehyde (3.3 g, 20.2 mmol) in benzene (30 mL) was placed into a round-bottomed flask equipped with a Dean-Stark trap. Acetic acid (115 μL, 2.0 mmol) and piperidine (200 μL, 2.0 mmol) were added. The mixture was heated at reflux with removal of azeotroped water for 12 h and the resulting mixture was diluted with ether and washed with water and brine. The organic layer was dried over anhydrous Na₂SO₄, and the solvent was removed by rotary evaporation. The product was purified by column chromatography (1:10 EtOAc/hexane) to give A (3.8 g, 66%) as a yellow oil. The NMR data showed the material to be composed of a 2:1 mixture of isomers. MS (M+H⁺, m/z) 291.

Compound B. To a solution of A (3.8 g, 13.2 mmol) in i-PrOH (30 mL) was added 2-amidinopyridinium chloride (2 g, 12.4 mmol) and sodium acetate (123 mg, 1.50 mmol). The mixture was heated at reflux for 12 h, and was then cooled, evaporated, and dissolved in a 1:1 mixture of 0.5 M HCl (aq)/EtOAc (60 mL). The organic layer was extracted with 1 M HCl (20 mL). The combined aqueous layers were washed with ether, rendered basic with ammonia solution (36 wt %), and extracted with EtOAc (3×50 mL). The combined organic layers were washed with water and brine, dried over anhydrous Na₂SO₄, and evaporated. The product was purified by column chromatography (1:5 EtOAc/hexane) to give B (2.6 g, 50%) as a yellow solid. MS (M+H⁺, m/z) 394.

Compound B-120. To a solution of B (30 mg, 0.076 mmol) in DMF (1 mL) was added triethylamine (60 μL, 0.43 mmol) followed by 1,4-diazepan-5-one (45 mg, 0.40 mmol). The mixture was stirring for 24 h at room temperature. The resulting mixture was diluted with EtOAc and washed with brine. The organic layer was dried over Na₂SO₄, and evaporation. The product was purified by column chromatography (1:3 EtOAc/hexane) to give 12 (88% yield) as a yellow solid. MS (M+H⁺, m/z) 472.

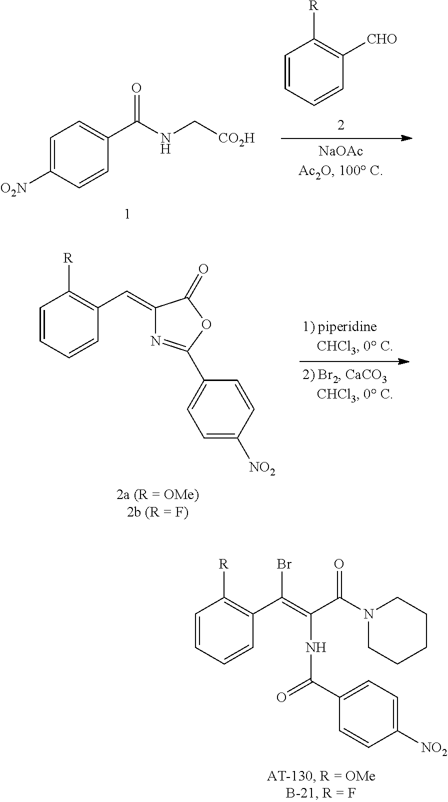

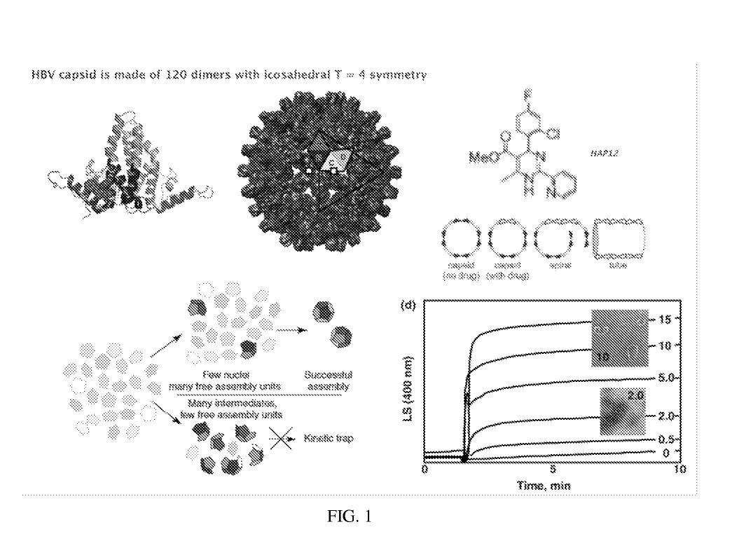

Synthesis of a Representative Propenamide Compound. Compounds AT-130 and B-21 were synthesized following a previously reported procedure as shown in FIG. 1. Condensation of appropriate benzaldehydes with 4-nitrohippuric acid (1) in the presence of sodium acetate in acetic anhydride at 100° C. provided the oxazalone intermediates (2), which were ring-opened with piperdine and subsequently brominated. Representative procedures and characterization data are as follows. (Z)-4-(2-Methoxybenzylidene)-2-(4-nitrophenyl) Oxazol-5(4H)-one (2a). 4-Nitrohippuric acid (1, 0.5 g, 2.23 mmol), o-anisaldehyde (0.276 g, 2.23 mmol), sodium acetate (0.183 g, 2.23 mmol), and acetic anhydride (0.6 mL) were combined and heated on a hot plate until the mixture just began to boil. It was then transferred to an oil bath and heated just below the boiling point for 1 h. Hot ethanol (2 mL) was added, and the mixture was stirred until homogeneous and was then cooled to RT. The resulting solid was collected by suction filtration, washed with a minimum quantity of cold ethanol and then with boiling water (approximately 1 mL), and dried in vacuo to give 2a (0.340 g, 68%). (E)-N-(1-Bromo-1-(2-methoxyphenyl)-3-oxo-3-(piperidin-1-yl)prop-1-en-2-yl)-4-nitrobenzamide (AT-130). To a solution of oxazolone 2a (0.5 g, 1.54 mmol) in chloroform at 0° C. was added dropwise a solution of piperidine (0.129 g, 1.54 mmol) in chloroform (1 mL). The yellow solution was stirred at 0° C. for 1 h. Solid calcium carbonate (0.154 g, 1.54 mmol) was added, followed by dropwise addition of bromine (0.246 g, 1.54 mmol) in chloroform (2 mL). The suspension was filtered to remove calcium salts, and the resulting solution was evaporated to dryness. The resulting orange oil was recrystallized from ethanol/water (4:1) to give compound AT-130 (0.312 g, 67%) as a colorless powder. 1H NMR (CDCl3): δ 0.53-1.44 (m, 4H), 3.30-3.35 (m, 4H), 7.75 (br, 1H, NH), 6.91-7.37 (m, 4H), 7.86-8.44 (m, 4H). (E)-N-(1-Bromo-1-(2-fluorophenyl)-3-oxo-3-(piperidin-1-yl)prop-1-en-2-yl)-4-nitrobenzamide (B-21). Prepared by the analogous procedure starting with 2 fluorobenzaldehyde. The final compound was isolated as a colorless powder in 70% yield. 1H NMR (CDCl3): δ 0.53-1.44 (m, 4H), 3.30-3.35 (m, 4H), 7.75 (br, 1H, NH), 7.11-7.39 (m, 4H), 7.97-8.45 (m, 4H).

### Methods

### Antiviral Activity

Antiviral activity was measured using an inducible HBV expression system, AD38 cells. (Ladner, S. K. et al., Antimicrob Agents Chemother 41, 1715-20 (1997)). Initial experiments tested the activity of 10 μM HAP (percentage viral assembly at 24 hour). For active molecules, effective concentrations were determined for suppression of HBV production by 50% and by 90%; this value is reported in μM. Compound toxicity was tested in the parent cell line, HepG2. This is reported as the concentration required to suppress cell growth by 50%, CC50 (in μM) and as the ratio of CC50/EC50, also known as the therapeutic index. As a control and for comparison, the results for the nucleoside analog 3TC (lamivudine) also described.

The activity of the hetero-aryl-dihydropyrimidine HAP12 compound on capsid-associated HBV-DNA (TaqMan real-time PCR), cccDNA (TaqMan real-time PCR) (Werle-Lapostolle, Gastroenterology 2004) and pgRNA levels (quantitative realtime PCR) (Belloni, PNAS 2009) were assessed in three in vitro HBV replication models:

a) HepG2 cells transfected with a linear full-length genotype A HBV DNA (Gunther, J Virol 1995; Pollicino, Gastroenterology 2006);

b) the HepG2 H1.3 genotype D HBV stable clone that accumulates cccDNA when cultured in “conditioned” medium at high confluence (Protzer, Gastroenterology 2007; Lucifora, J Hepatol 2011);

c) the AD38 cells are a stable HepG2-derived HBV genotype D clone that, upon tetracycline removal, expresses pgRNA,

assembles subviral particles in the cytoplasm, accumulates cccDNA in the nucleus and secretes HBV virions in the cell supernatant (Ladner, J Virol 1997).

Recruitment of HBc and cccDNA bound histones modifications were assessed using the cccDNA ChIP assay (Pollicino, 2006)

CpAMs were used to treat a transient transfection of huh7 cells. Cytoplasmic RNA was harvested and quantified by RT-per. The transfection system is described by Lentz and Loeb (Lentz and Loeb (2010) J Virol Methods 169, 52-60.) Fresh medium with drug was added to cultured cells daily for four days at concentrations from 0.001 to 10 micromolar. The two points at 0.00001 are both controls where there was no drug and no DMSO. The controls and the DMSO-treated cells had similar amounts of RNA. The treated samples showed that high concentrations of HAP12 were needed to suppress RNA by 50% relative to the ability of the same drug to suppress secreted DNA synthesis. For comparison, the respective EC50s for DNA suppression for HAP12, HAP13 and AT130 are 12 nM, 6.1 μM, and 2.4 μM while the EC50s for RNA suppression was 0.5, 5.0, and 0.5 μM. (See FIG. 7)

1. A method for treating a patient having an infection by hepatitis B virus (HBV), the method comprising the step of administering to the patient a therapeutically effective amount a compound capable of inhibiting accumulation of HBV pgRNA in an HBV infected cell of the patient.

2. The method of claim 1 wherein the therapeutically effective amount is greater than or equal to that corresponding to an in vitro dose that is 10-fold greater than required to suppress DNA synthesis or 1 μM in a cell-based assay of pgRNA accumulation in HepG2 H1.3 cells.

3. The method of claim 1 wherein the compound has the formula 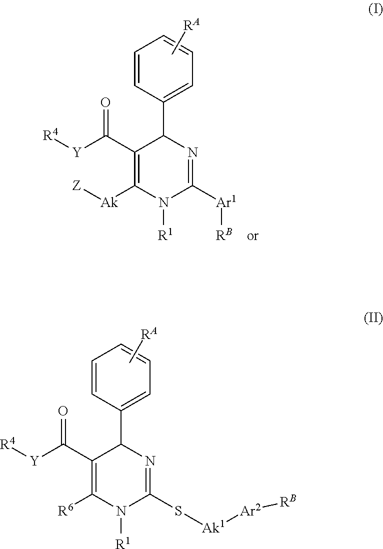
wherein
Ar¹ and Ar² are each independently selected from aryl or heteroaryl;
R¹ is hydrogen or a pro-drug forming group;
Ak is alkylene;
Ak¹ is (CH₂)n, where n is 1 to 4;
Z is hydrogen or 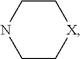
where X is CHN₃, C═O, C═NR⁵, —C(O)N(R^{N})—, or NR^{N}, where R⁵ is hydroxy or a derivative thereof or amino or a derivative thereof, and R^{N} is selected from the group consisting of alkyl, alkenyl, alkynyl, heteroalkyl, arylalkyl, heteroarylalkyl, alkyl-C(O), heteroalkyl-C(O), alkoxyl-C(O), alkynyl-C(O), alkylacylamino-C(O), and heteroalkylacylamino-C(O), each of which is optionally substituted;
R⁴ is alkyl, heteroalkyl, alkenyl, or alkynyl, each of which is optionally substituted;
Y is O, or HN;
R^{A} represents from 0 to 3 substituents independently selected in each instance from the group consisting of halo, and alkyl, heteroalkyl, aryl, heteroaryl, amino and derivatives thereof, and hydroxyl and derivatives thereof, each of which is optionally substituted; and
R^{B} represents from 0 to 3 substituents independently selected in each instance from the group consisting of halo, and alkyl, heteroalkyl, aryl, heteroaryl, amino and derivatives thereof, and hydroxyl and derivatives thereof, each of which is optionally substituted; and
R⁶ is in each instance independently selected from the group consisting of hydrogen and Ak-Z¹, where Ak is alkylene, and Z¹ is hydrogen or NR²R³; where R² and R³ are independently in each instance selected from the group consisting of hydrogen, and alkyl, cycloalkyl, heteroalkyl and heterocycloalkyl, each of which is optionally substituted, or
R² and R³ are taken together with the attached nitrogen to form 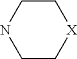
wherein X is CHN₃, C═O, —C(O)N(R^{Na})—, C═NR⁵, or NR^{Na}; where R⁵ is hydroxy or a derivative thereof or amino or a derivative thereof; and R^{Na} is selected from the group consisting of hydrogen, and alkyl, alkenyl, alkynyl, heteroalkyl, arylalkyl, heteroarylalkyl, alkyl-C(O), heteroalkyl-C(O), alkoxyl-C(O), alkynyl-C(O), alkylacylamino-C(O), and heteroalkylacylamino-C(O), each of which is optionally substituted.

4. A method for identifying a compound useful for the treatment of infection hepatitis B virus (HBV), comprising:
contacting a cell infected with HBV with a test compound in a culture medium, or administering a potential compound to an animal;
retrieving a sample from the cell, the culture medium, or from tissue of the animal, at one or more time points;
analyzing the sample for one or more attributes selected from the group consisting of HBV cccDNA concentration, amount of methylated cccDNA, acetylation state of cccDNA, HBV cccDNA transcription, HBV RNA concentration in cellular cytoplasm, HBV RNA concentration in the cell nucleus, concentration of unassembled capsid protein, and HBV S antigen concentration; and
identifying the compound as useful for treating hepatitis B based on the reduction or increase of one of more of the attributes.

5. The method of claim 4, wherein the analyzing step comprises analyzing HBV capsid stabilization or capsid nucleation.

6. The method of claim 4, wherein the HBV RNA concentration is selected from pgRNA, subgeneric subgenomic RNA, or spliced RNA.

7. The method of claim 4 wherein the analyzing step comprises determining the effect the compound on capsid stability, the effect of the compound on nucleation of assembly, the affinity of the compound for capsid, the affinity of the compound for dimer, or the ability of the compound to induce allosteric effect.

8. The method of claim 4, further comprising varying the concentration of the potential compound until one or more of the attributes is reduced or increased.

9. The method of claim 4, wherein the candidate compound is heteroaryldihydropyrimidine compound.

10. The method of claim 4, wherein the candidate compound has the formula 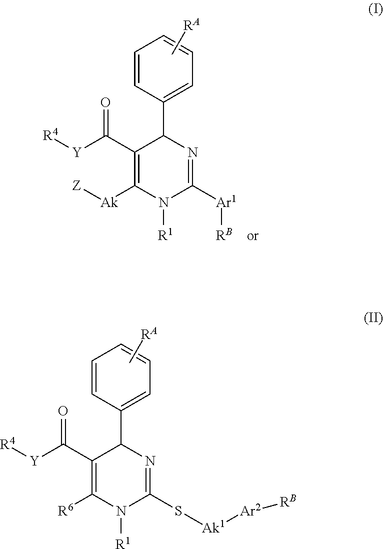
wherein
Ar¹ and Ar² are each independently selected from aryl or heteroaryl;
R¹ is hydrogen or a pro-drug forming group;
Ak is alkylene;
Ak¹ is (CH)n, where n is 1 to 4;
Z is hydrogen or 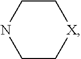
where X is CHN₃, C═O, C═NR⁵, —C(O)N(R^{N})—, or NR^{N}, where R⁵ is hydroxy or a derivative thereof or amino or a derivative thereof, and R^{N} is selected from the group consisting of alkyl, alkenyl, alkynyl, heteroalkyl, arylalkyl, heteroarylalkyl, alkyl-C(O), heteroalkyl-C(O), alkoxyl-C(O), alkynyl-C(O), alkylacylamino-C(O), and heteroalkylacylamino-C(O), each of which is optionally substituted;
R⁴ is alkyl, heteroalkyl, alkenyl, or alkynyl, each of which is optionally substituted;
Y is O, or HN;
R^{A} represents from 0 to 3 substituents independently selected in each instance from the group consisting of halo, and alkyl, heteroalkyl, aryl, heteroaryl, amino and derivatives thereof, and hydroxyl and derivatives thereof, each of which is optionally substituted; and
R^{B} represents from 0 to 3 substituents independently selected in each instance from the group consisting of halo, and alkyl, heteroalkyl, aryl, heteroaryl, amino and derivatives thereof, and hydroxyl and derivatives thereof, each of which is optionally substituted; and
R⁶ is in each instance independently selected from the group consisting of hydrogen and Ak-Z¹, where Ak is alkylene, and Z¹ is hydrogen or NR²R³; where R² and R³ are independently in each instance selected from the group consisting of hydrogen, and alkyl, cycloalkyl, heteroalkyl and heterocycloalkyl, each of which is optionally substituted, or
R² and R³ are taken together with the attached nitrogen to form 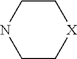
wherein X is CHN₃, C═O, —C(O)N(R^{Na})—, C═NR⁵, or NR^{Na}; where R⁵ is hydroxy or a derivative thereof or amino or a derivative thereof; and R^{Na} is selected from the group consisting of hydrogen, and alkyl, alkenyl, alkynyl, heteroalkyl, arylalkyl, heteroarylalkyl, alkyl-C(O), heteroalkyl-C(O), alkoxyl-C(O), alkynyl-C(O), alkylacylamino-C(O), and heteroalkylacylamino-C(O), each of which is optionally substituted.

11. The method of claim 8, wherein the concentration of the potential compound is about 1 μM to about 10 μM or from about 0.1 μM to about 1 μM.

12. The method of claim 4, wherein the tissue is liver tissue.

13. The method of claim 4, wherein the animal is a rodent or human.

14. The method of claim 4, wherein the cell is from a cell line derived from human hepatocytes (e.g. Huh7, AD38, HepG2, or HepG2.2.15).

15. A method of reducing pgRNA transcription in a HBV infected cell, comprising contacting the cell with a heteroaryldihydropyrimidine compound.

16. A method of reducing pgRNA transcription in a HBV infected cell, comprising contacting the cell with the compound described in claim 3.

17. The method of claim 3 wherein R^{A} represents 2-cholor-4-fluoro.

18. The method of claim 3 wherein Ar¹ is 2-pyridyl.

19. The method of claim 3 wherein R^{B} represents 0 substituents.

20. The method of claim 3 wherein Y is O.

21. The method of claim 3 wherein R⁴ is methyl.

22. The method of claim 3 where in Ak is methylene.

23. The method of claim 3 wherein Z is hydrogen.

24. The method of claim 3 wherein Z-Ak is CH₃.

25. The method of claim 22 wherein X is C═O, —C(O)N(R^{N})—, or NR^{N}.

26. The method of claim 25 wherein Xis C═O.

27. The method of claim 3 wherein R⁶ is methyl.

28. The method of claim 3 wherein R⁶ is 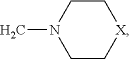
where X is C═O, —C(O)N(R^{Na})—, or NR^{Na}, where R^{Na} is hydrogen or alkyl, alkenyl, alkynyl, heteroalkyl, arylalkyl, heteroarylalkyl, alkyl-C(O), heteroalkyl-C(O), alkylacylamino-C(O), and heteroalkylacylamino-C(O), each of which is optionally substituted.

29. The method of claim 28 wherein X is C═O.
